(19) United States
(12) Patent Application Publication
(10) Pub. No.: US 2016/0038201 A1
(43) Pub. Date: Feb. 11, 2016

(54) FLEXIBLE SCREW AND METHODS FOR SYNDESMOSIS REPAIR

(71) Applicant: Wright Medical Technology, Inc., Memphis, TN (US)

(72) Inventor: Shannon D. Cummings, Hernando, MO (US)

(21) Appl. No.: 14/402,930

(22) PCT Filed: Aug. 11, 2014
(86) PCT No.: PCT/US14/50560

§ 371 Date: Nov. 21, 2014

(51) Int. Cl. A61B 17/84; A61B 17/86

(52) U.S. Cl. CPC A61B 17/842; A61B 17/866; A61B 17/8615; A61B 17/863; A61B 17/864

(57) ABSTRACT

A flexible screw comprising a first end and a second end coupled by a flexible mesh is disclosed. The first end comprises a threaded section. The second end comprises a head. The flexible mesh defines a hollow cylinder. A first driver engagement channel is defined by the head and extends from a proximal end of the head to the flexible mesh. A second driver engagement channel is defined by the threaded section and extends from the flexible mesh to a first depth within the threaded section. The first driver engagement channel and the second driver engagement channel are configured to receive a driver therein.

### FIELD OF THE INVENTION

This disclosure generally relates to systems and methods for orthopedic surgery. More particularly, this disclosure relates to systems and methods for syndesmosis repair.

### BACKGROUND

Syndesmosis is a slightly moveable articulation where bony surfaces are coupled by an interossesous ligament. An example of a syndesmosis is the tibiofibular articulation syndesmosis between the tibia and the fibula. Syndesmosis may be torn as a result of bone fractures or other trauma.

Current syndesmosis repair systems and methods rely on rigid screws for coupling the tibia and the fibular and replacing the syndesmosis. The rigid screw inhibits normal movement and articulation of the bones, for example, the tibia and fibula, and further limits one or more corresponding joints. Current systems fail to provide the required flexibility to maintain the natural flexibility and lateral movement of the bones.

### SUMMARY

The present subject matter relates to a flexible screw for syndesmotic repair, as well as methods of inserting the flexible screw into bones. The flexible screw has a number of different embodiments, each of which correspond to different nuances in their respective methods of insertion. All of the flexible screws disclosed herein comprise a first end and a second end coupled by a flexible mesh is disclosed. The first end comprises a threaded section. The second end comprises a head. The flexible mesh defines a hollow cylinder. A first driver engagement channel is defined by the head and extends from a proximal end of the head to the flexible mesh.

In some embodiments, a system comprising a flexible screw and a driver is disclosed. The flexible screw comprises a first end and a second end coupled by a flexible mesh is disclosed. The first end comprises a threaded section. The second end comprises a head. The flexible mesh defines a hollow cylinder. A first driver engagement channel is defined by the head and extends from a proximal end of the head to the flexible mesh. The driver is sized and configured to be received within the first driver engagement channel.

In some embodiments, a method of syndesmotic repair is disclosed. In a first step, a flexible screw is located proximal to a first bone. The flexible screw comprises a first end having a threaded section, a second end having a head, and a flexible mesh coupling the first end and the second end along a longitudinal axis. In a second step, a driver is inserted into a first driver engagement channel defined by the head of the flexible screw. In a third step, the flexible screw is rotated such that the flexible screw is driven through the first bone and into the second bone. The flexible screw is driven to a predetermined depth within the second bone.

### BRIEF DESCRIPTION OF THE DRAWINGS

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 illustrates one embodiment of a screw comprising a first end and a second end coupled by a flexible mid-section.

FIG. 2 illustrates a cross-sectional view of the screw of FIG. 1.

FIG. 3 illustrates one embodiment of a screw comprising a flexible mid-section and a driver configured to couple thereto.

FIG. 4 illustrates one embodiment of the screw of FIG. 3 having the driver inserted therein.

FIG. 5 illustrates a cross-sectional view of the screw of FIG. 4.

FIG. 6 illustrates one embodiment of a screw comprising a flexible mid-section coupling a first bone and a second bone.

FIG. 7 illustrates one embodiment of a method for syndesmosis repair using a flexible screw.

### DETAILED DESCRIPTION

The description of the exemplary embodiments is intended to be read in connection with the accompanying drawings, which are to be considered part of the entire written description. In the description, relative terms such as “lower,” “upper,” “horizontal,” “vertical,” “proximal,” “distal,” “above,” “below,” “up,” “down,” “top” and “bottom,” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

The present disclosure generally provides a flexible screw for syndesmosis repair and/or stabilization. The flexible screw generally comprises a first end comprising a threaded section and a second end comprising a head coupled by a flexible mid-section. The mid-section comprises a metal mesh material configured to maintain a desired tensile strength while providing flexibility between a first bone and a second bone.

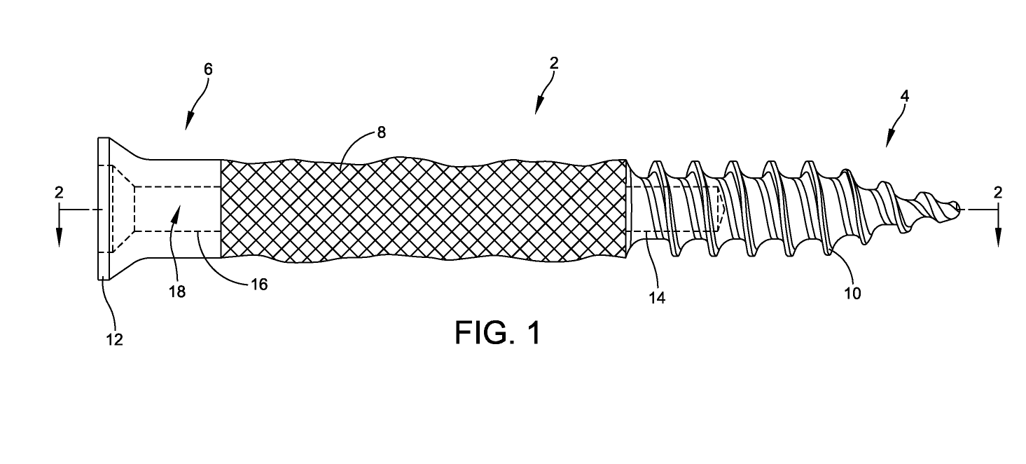

FIG. 1 illustrates one embodiment of a flexible screw 2 comprising a first end 4 and a second end 6 coupled by a flexible mid-section 8. The first end 4 comprises a threaded section 10. The threaded section 10 is configured to be inserted through a first bone and into a second bone to anchor the first bone to the second bone. The threaded section 10 may comprise threads suitable for cancellous and/or cortical bone anchoring. The threaded section 10 may define a distal driver channel 14. The distal driver channel 14 is configured to receive a driver therein. The distal driver channel 14 extends from a proximal end of the threaded section 10 distally to a predetermined depth in the threaded section 10. In some embodiments the threaded section 10 comprises a self-drilling and/or self-tapping thread. In other embodiments, the threaded section 10 is configured to engage a pre-drilled and/or pre-tapped hole. The threaded section 10 may comprise any suitable material, such as, for example, titanium.

The second end 6 comprises a metal head 12. The metal head 12 defines a proximal driver channel 16. The proximal driver channel comprises a longitudinal channel extending from a proximal opening 20 of the metal head 12 to the distal side of the metal head 12. The proximal driver channel 16 is configured to receive a driver therethrough. The proximal driver channel 16 may be configured to engage a second portion of a driver, such as, for example, a second portion of a hex driver. The metal head 12 may comprise any suitable material to provide an interface to the driver, such as, for example, titanium.

The flexible mid-section 8 comprises a flexible metal mesh. The flexible mid-section 8 is configured to provide flexibility to the flexible screw 2 while maintaining a required tensile strength for coupling a first bone and a second bone. In some embodiments, the flexible mid-section 8 comprises a cylinder defining a hollow column extending longitudinally therethrough. The flexible metal mesh may comprise, for example, a titanium mesh.

In some embodiments, the proximal driver channel 16, flexible mid-section 8, and the distal driver channel 14 define a continuous driver channel 18. The driver channel 18 extends from a proximal opening 20 in the metal head 12, through the proximal driver channel 16 and the flexible mid-section 8, and into the distal driver channel 14. The driver channel 18 is configured to receive a driver therein. A driver inserted into the driver channel 18 engages the first end 4 and the second end 6 of the flexible screw 2 to prevent twisting the flexible metal mesh 8 during insertion of the flexible screw 2.

In some embodiments, the flexible screw 2 is configured to engage cancellous and/or cortical bone. The threaded section 10 may comprise any predetermined thread pattern to engage a bone and may comprise a self-drilling and/or a self-tapping thread. The threaded section 10 may comprise a length sufficient to engage a bone. For example, in some embodiments, the threaded section 10 comprises a threaded section having a length of 3.5-4.0 mm. In some embodiments, the length of the threaded section 10 corresponds to a thickness of a bone anchored by the threaded section 10.

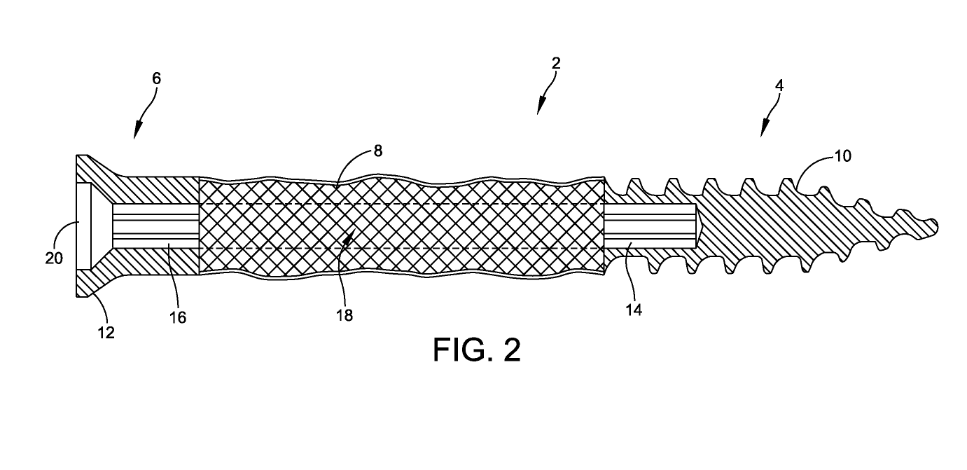

FIG. 2 illustrates a cross-sectional view of the flexible screw 2 of FIG. 1. As shown in FIG. 2, the channel 18 comprises a proximal driver channel 16 in the metal head 12 and the distal driver channel 14 in the threaded section 10. The proximal driver channel 16 and the distal driver channel 14 are coupled through the hollow interior of the flexible metal mesh 8. A proximal opening 20 comprising sloped sides is configured to receive a driver therein and direct the driver into the proximal driver channel 16. The proximal driver channel 16 aligns the driver with the distal driver channel 14 during insertion. The proximal driver channel 16 and the distal driver channel 14 comprise internal geometries configured to match the geometry of a driver inserted therein.

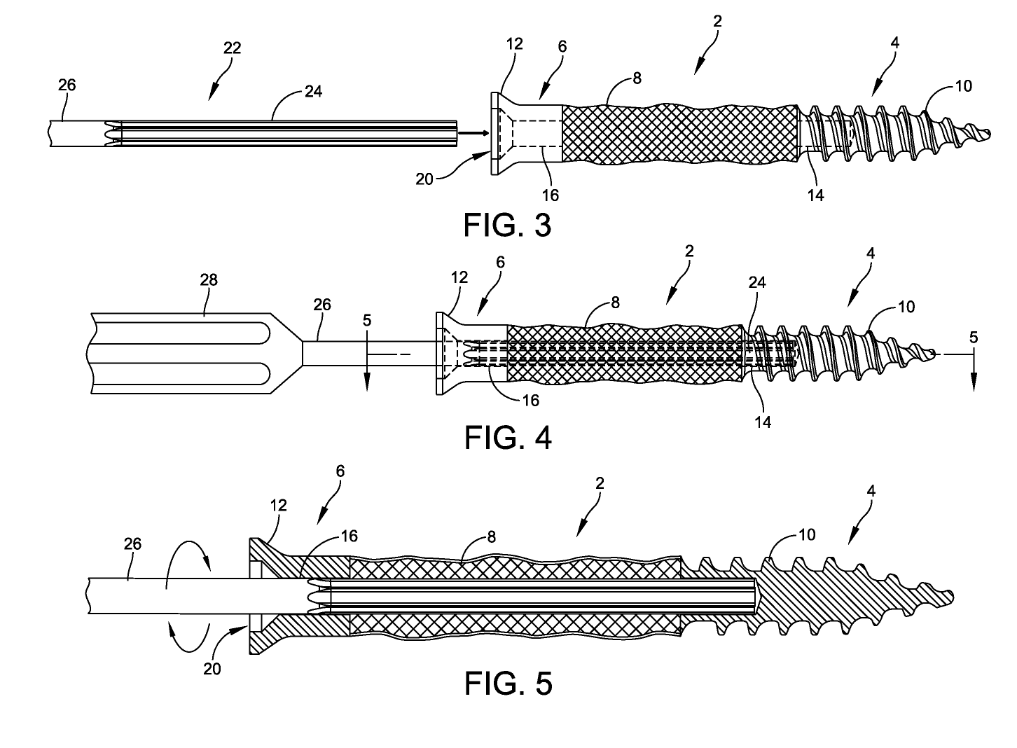

FIG. 3 illustrates one embodiment of a flexible screw 2 and a driver 22. The driver 2 comprises a driving section 24 and a shaft 26. The shaft 26 extends proximally to a handle 28. The driver 22 is sized and configured to be received within the proximal driver channel 16 and the distal driver channel 14. In some embodiments, the driving section 24 comprises a distal driving section and a proximal driving section coupled by a smooth shaft. In some embodiments, the driving section 24 comprises a continuous driving section having a length sufficient to extend from the proximal opening 20 of the metal head 12 into the distal driver channel 14.

FIG. 4 illustrates the flexible screw 2 of FIG. 3 having the driver 22 inserted therein. The driving section 24 of the driver 22 comprises a length such that the driving section 24 extends from the proximal driver channel 16 to the distal driver channel 14. A handle 28 is coupled to the shaft 26 to allow an operator to rotate the driver 22 to drive the flexible screw 2 into a bone. By engaging both the metal head 12 and the threaded section 10 of the screw, the driver 22 prevents the flexible mid-section 8 from twisting during insertion.

FIG. 5 illustrates a cross-sectional view of the flexible screw 2 of FIG. 3 having the driver 22 inserted therein. The driver 22 is inserted through the proximal opening 20 in the metal head 12, through the proximal driver channel 22 in the metal head 12, the hollow middle of the flexible mesh 8, and into a distal driver channel 14 formed in the threaded section 10. The distal driver channel 14 extends at least partially into the first end 4. By engaging both the metal head 12 and the threaded section 10 simultaneously, the driver 22 rotates the metal head 12 and the threaded section 10 in unison, preventing twisting of the flexible mesh 8 during insertion of the flexible screw 2.

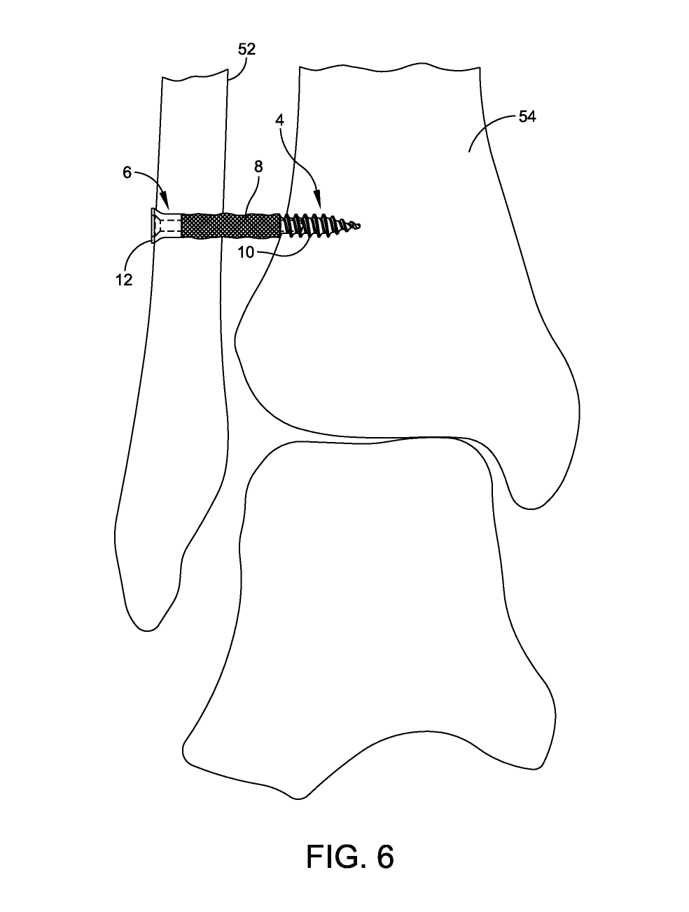

In some embodiments, the flexible screw 2 is configured for use in syndesmotic repair and fixation. FIG. 6 illustrates one embodiment of the flexible screw 2 coupling a first bone 52 and a second bone 54. The first bone 52 and the second bone 54 may be joined through the syndesmosis 56 between the first bone 52 and the second bone 54. The flexible screw 2 provides a required tensile strength for syndesmotic fixation while providing for the necessary proximal/distal motion of the first bone 52 and the second bone 54. In some embodiments, the first bone 52 comprises a tibia and the second bone 54 comprises a fibula. As shown in FIG. 6, the flexible screw 2 extends from a first, or proximal, side of a first bone 52, through the first bone and out of a second, or distal side, of the first bone 52, across a syndesmosis between the first bone 52 and a second bone 54, and at least partially into a proximal side of the second bone 54. In some embodiments, the flexible mid-section 8 comprises a length sufficient to bridge the gap between the first bone 52 and the second bone 54. In some embodiments, the length of the flexible mid-section 8 corresponds to a lateral width of the syndesmosis between the first bone 52 and the second bone 54.

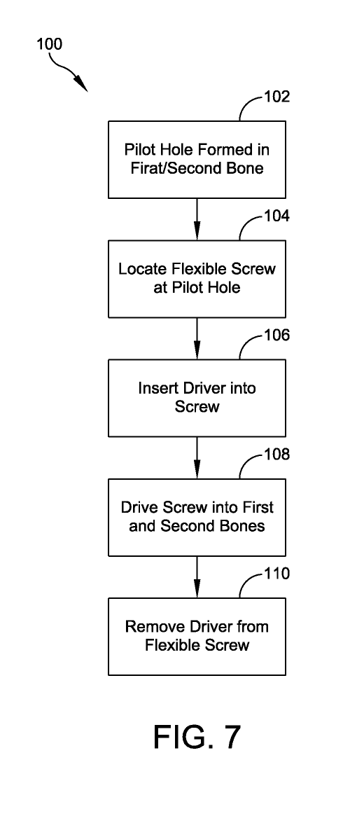

FIG. 7 is a flowchart illustrating one embodiment of a method 100 for performing syndesmotic repair between a first bone and a second bone. In some embodiments, in a first step 102, a pilot hole is formed in one of a first bone 52 and/or a second bone 54. The pilot hole may be formed by, for example, a k-wire, a drill, and/or any other suitable device. In a second step 104, a flexible screw 2 is aligned with the first bone 52. The flexible screw 2 comprise a first driver engagement channel 16 defined by a head 12 and a second driver engagement channel 14 defined by a threaded section 10. In a third step 106, a driver 22 is inserted into the first and second driver engagement sections 14, 16 of the flexible screw 2. The driver 22 engages a proximal channel 16 and a distal channel 14 formed in the flexible screw 2. In some embodiments, the flexible screw 2 comprises a self-drilling and/or self-tapping thread. In other embodiments, the pilot hole comprises a pre-drilled and/or pre-tapped hole.

In a fourth step 108, the driver 22 is rotated by a clinician to drive the flexible screw 2 through the first bone 52 and into the second bone 54. The flexible screw 2 is driven to a predetermined depth within the second bone 54. In some embodiments, the predetermined depth corresponds to a length of the threaded section 10. In some embodiments, a flexible section 8 of the flexible screw 2 comprises a length such that the flexible section 8 extends into partially into the first bone 52 and/or the second bone 54 when the flexible screw 2 is installed. In other embodiments, a solid section (not shown) may couple the flexible mesh 8 to the metal head 12 such that the flexible mesh 8 extends from a distal side of the second bone 54 to a proximal side of the first bone 52. The flexible screw 2 provides tensile strength sufficient to maintain the syndesmotic fixation between the first bone 52 and the second bone 54 while allowing natural movement of the first bone 52 and the second bone 54. The flexible screw 2 allows for the necessary proximal/distal motion of the first bone 52 and the second bone 54 while maintaining the proper later and medial gap. The method 100 further comprises a step 110 of removing the driver 22 from the flexible screw 2 The driver 22 is withdrawn proximally from the flexible screw 2. Once the driver 22 is removed, the flexible section 8 is allowed to flex and/or move to allow natural movement of the first bone 52 and the second bone 54.

In some embodiments, a flexible screw is disclosed. The flexible screw comprises a first end and a second coupled by a flexible mesh. The first end comprises a threaded section. The second end comprises a head.

In some embodiments, the flexible mesh defines a hollow cylinder.

In some embodiments, the head defines a first driver engagement channel configured to receive a driver therein. The first driver engagement channel extends from a proximal end of the head to the flexible mesh.

In some embodiments, the threaded section defines a second driver engagement channel configured to receive the driver therein. The second driver engagement channel extends from the flexible mesh to a predetermined depth within the threaded section. The first driver engagement channel and the second drive engagement channel are coupled through the hollow cylinder of the flexible mesh.

In some embodiments, the head comprises titanium.

In some embodiments, the threaded section comprises a titanium thread.

In some embodiments, the threaded section comprises a 3.5-4.0 mm thread.

In some embodiments, the flexible mesh comprises a titanium mesh.

In some embodiments, a system for bone fixation is disclosed. The system comprises a flexible screw and a driver. The flexible screw comprises a first end and a second end coupled by a flexible mesh. The first end comprises a threaded section. The second end comprises a head defining a first driver engagement channel. The flexible mesh extends along a longitudinal axis. The driver is sized and configured to be received within the first driver engagement channel.

In some embodiments, the flexible mesh defines a hollow cylinder.

In some embodiments, the threaded section defines a second driver engagement channel sized and configured to receive the driver therein. The first driver engagement channel extends from a proximal end of the head to the flexible mesh and the second driver engagement channel extends from the flexible mesh to a predetermined depth within the threaded section. The first driver engagement channel and the second driver engagement channel are axially aligned.

In some embodiments, the head of the flexible screw comprises titanium.

In some embodiments, the flexible mesh comprises a titanium mesh.

In some embodiments, the threaded section comprises a titanium thread.

In some embodiments, the threaded section comprises a 3.5-4.0 mm thread.

In some embodiments, the driver comprises a hex driver and the first and second engagement channels comprise complementary hex engagement channels.

In some embodiments, a method of syndesmotic fixation is disclosed. The method comprises locating a flexible screw proximal to a first bone, inserting a driver into a driver engagement channel in the flexible screw, and rotating the flexible screw to drive the flexible screw through the first bone and into a second. The flexible screw comprises a first end and a second end coupled by a flexible mesh. The first end comprises a threaded section. The second end comprises a head. The flexible mesh extends along a longitudinal axis. The first driver engagement channel is defined by the head.

In some embodiments, the driver is inserted into a second driver engagement channel defined by the threaded section. The driver extends through the first driver engagement channel, the flexible mesh, and into the second driver engagement channel.

In some embodiments, the first bone comprises a fibula and the second bone comprises a tibia.

In some embodiments, the driver is removed from the first and second engagement channels after the flexible screw is driven into the second bone.

Although the subject matter has been described in terms of exemplary embodiments, it is not limited thereto. Rather, the appended claims should be construed broadly, to include other variants and embodiments, which may be made by those skilled in the art.

What is claimed is:

1. A screw, comprising:
a first end comprising a threaded section;
a second end comprising a head; and
a flexible mesh coupling the first end and the second end.

2. The screw of claim 1, wherein the flexible mesh defines a hollow cylinder.

3. The screw of claim 2, wherein the head defines a first driver engagement channel configured to receive a driver therein, wherein the first driver engagement channel extends from a proximal end of the head to the flexible mesh.

4. The screw of claim 3, wherein the threaded section defines a second driver engagement channel configured to receive the driver therein, wherein the second driver engagement channel extends from the flexible mesh to a predetermined depth within the threaded section.

5. The screw of claim 1, wherein the head comprises titanium.

6. The screw of claim 1, wherein the threaded section comprises a titanium thread.

7. The screw of claim 6, wherein the threaded section comprises a 3.5-4.0 mm thread.

8. The screw of claim 1, wherein the flexible mesh comprises a titanium mesh.

9. A system for bone fixation, comprising:
a flexible screw comprising:
a first end comprising a threaded section;
a second end comprising a head defining a first driver engagement channel;
a flexible metal coupling the first end and the second end on a longitudinal axis; and
a driver sized and configured to be received within the driver engagement channel.

10. The system for bone fixation of claim 9, wherein the flexible mesh comprises a hollow cylinder.

11. The system for bone fixation of claim 10, wherein the threaded section defines a second driver engagement channel sized and configured to receive the driver therein, wherein the first driver engagement channel extends from a proximal end of the head to the flexible mesh, wherein the second driver engagement channel extends from the flexible mesh to a predetermined depth within the threaded section, and wherein the first driver engagement channel and the second driver engagement channel are axially aligned.

12. The system of claim 11, wherein the head of the flexible screw comprises titanium.

13. The system of claim 11, wherein the flexible mesh comprises a titanium mesh.

14. The system of claim 11, wherein the threaded section comprises a titanium thread.

15. The system of claim 14, wherein the titanium thread comprises a 3.5-4.0 mm titanium thread.

16. The system of claim 11, wherein the driver comprises a hex driver and wherein the first driver engagement channel and the second driver engagement channel comprise hex engagement channels.

17. A method of syndesmotic fixation, comprising:
locating a flexible screw proximal to a first bone, the flexible screw comprising a first end having a threaded section, a second end having a head, and a flexible mesh coupling the first end and the second end along a longitudinal axis;
inserting a driver into a first driver engagement channel defined by the head of the flexible screw; and
rotating the flexible screw such that the flexible screw is driven through the first bone and into the second bone, wherein the flexible screw is driven to a predetermined depth within the second bone.

18. The method of claim 17, further comprising inserting the driver into a second driver engagement channel defined by the threaded section of the flexible screw, wherein the driver extends through the first driver engagement channel, the flexible mesh, and into the second driver engagement channel.

19. The method of claim 18, wherein the first bone comprises a fibula and the second bone comprises a tibia.

20. The method of claim 18, further comprising removing the driver from the first and second driver engagement channels.
